(19) United States
(12) Patent Application Publication
(10) Pub. No.: US 2019/0184121 A1
(43) Pub. Date: Jun. 20, 2019

(54) METHOD FOR OBTAINING FACIAL METRICS OF A PERSON AND INDENTIFYING A MASK FOR THE PERSON FROM SUCH METRICS

(71) Applicant: KONINKLIJKE PHILIPS N.V., EINDHOVEN (NL)

(72) Inventors: RICHARD THOMAS HAIBACH, VERONA, PA (US); DANIEL STEED, NORTH HUNTINGDON, PA (US); ROBERT WILLIAM BAIKO, PITTSBURGH, PA (US)

(21) Appl. No.: 16/220,032

(22) Filed: Dec. 14, 2018

Related U.S. Application Data
(60) Provisional application No. 62/607,475, filed on Dec. 19, 2017.

(51) Int. Cl. A61M 16/06; A61B 5/107; A61B 5/00; G06K 9/00

(52) U.S. Cl. CPC A61M 16/0605; A61B 5/1077; A61B 5/4818; A61M 16/0051; A61B 5/6803; G06K 9/00281; A61B 5/1079

(57) ABSTRACT

A method of determining facial metrics of a person includes: receiving a request that the person requires a mask; navigating an autonomously operable vehicle to the person using at least one imaging device; capturing a number of images of the person using the at least one imaging device; and determining facial metrics of the person from the number of images. The method may be further employed in a method for identifying a mask for the person which further includes determining a mask for the user from the facial metrics; and identifying the mask to the person.

### CROSS-REFERENCE TO RELATED APPLICATIONS

This patent application claims the priority benefit under 35 U.S.C. § 119(e) of U.S. Provisional Application No. 62/607,475, filed on Dec. 19, 2017, the contents of which are herein incorporated by reference.

### BACKGROUND OF THE INVENTION

### 1. Field of the Invention

The present invention pertains to methods for obtaining facial metrics of a person and further identifying a mask for the person from such metrics.

### 2. Description of the Related Art

Many individuals suffer from disordered breathing during sleep. Sleep apnea is a common example of such sleep disordered breathing suffered by millions of people throughout the world. One type of sleep apnea is obstructive sleep apnea (OSA), which is a condition in which sleep is repeatedly interrupted by an inability to breathe due to an obstruction of the airway; typically the upper airway or pharyngeal area. Obstruction of the airway is generally believed to be due, at least in part, to a general relaxation of the muscles which stabilize the upper airway segment, thereby allowing the tissues to collapse the airway. Another type of sleep apnea syndrome is a central apnea, which is a cessation of respiration due to the absence of respiratory signals from the brain's respiratory center. An apnea condition, whether obstructive, central, or mixed, which is a combination of obstructive and central, is defined as the complete or near cessation of breathing, for example a 90% or greater reduction in peak respiratory air-flow.

Those afflicted with sleep apnea experience sleep fragmentation and complete or nearly complete cessation of ventilation intermittently during sleep with potentially severe degrees of oxyhemoglobin desaturation. These symptoms may be translated clinically into extreme daytime sleepiness, cardiac arrhythmias, pulmonary-artery hypertension, congestive heart failure and/or cognitive dysfunction. Other consequences of sleep apnea include right ventricular dysfunction, carbon dioxide retention during wakefulness, as well as during sleep, and continuous reduced arterial oxygen tension. Sleep apnea sufferers may be at risk for excessive mortality from these factors as well as by an elevated risk for accidents while driving and/or operating potentially dangerous equipment.

Even if a patient does not suffer from a complete or nearly complete obstruction of the airway, it is also known that adverse effects, such as arousals from sleep, can occur where there is only a partial obstruction of the airway. Partial obstruction of the airway typically results in shallow breathing referred to as a hypopnea. A hypopnea is typically defined as a 50% or greater reduction in the peak respiratory air-flow. Other types of sleep disordered breathing include, without limitation, upper airway resistance syndrome (UARS) and vibration of the airway, such as vibration of the pharyngeal wall, commonly referred to as snoring.

It is well known to treat sleep disordered breathing by applying a continuous positive air pressure (CPAP) to the patient's airway. This positive pressure effectively “splints” the airway, thereby maintaining an open passage to the lungs. It is also known to provide a positive pressure therapy in which the pressure of gas delivered to the patient varies with the patient's breathing cycle, or varies with the patient's breathing effort, to increase the comfort to the patient. This pressure support technique is referred to as bi-level pressure support, in which the inspiratory positive airway pressure (IPAP) delivered to the patient is higher than the expiratory positive airway pressure (EPAP). It is further known to provide a positive pressure therapy in which the pressure is automatically adjusted based on the detected conditions of the patient, such as whether the patient is experiencing an apnea and/or hypopnea. This pressure support technique is referred to as an auto-titration type of pressure support, because the pressure support device seeks to provide a pressure to the patient that is only as high as necessary to treat the disordered breathing.

Pressure support therapies as just described involve the placement of a patient interface device including a mask component having a soft, flexible sealing cushion on the face of the patient. The mask component may be, without limitation, a nasal mask that covers the patient's nose, a nasal/oral mask that covers the patient's nose and mouth, or a full face mask that covers the patient's face. Such patient interface devices may also employ other patient contacting components, such as forehead supports, cheek pads and chin pads. The patient interface device is typically secured to the patient's head by a headgear component. The patient interface device is connected to a gas delivery tube or conduit and interfaces the pressure support device with the airway of the patient, so that a flow of breathing gas can be delivered from the pressure/flow generating device to the airway of the patient.

In order to optimize treatments as well as patient compliance with such treatments it is important to provide the patient with a well fit mask. As no two patient's faces are exactly the same, the best way to ensure an optimum fit is to provide the patient a custom/semi-custom mask that is sized/designed according to their specific facial geometry. Such custom/semi-custom CPAP masks require a scan of the patient's face. The scan is a critical element to generating the custom geometry. In order to gather the geometry of the patient's face, a camera or scanner is required. Current scanner technologies require an expensive setup comprised of a fixture with more than one camera. Hand held 3-D scanners today are currently extremely expensive. As a result of the cost of such devices, access to such devices is generally limited. Also, those who would most benefit from treatments for disordered breathing are commonly unable to travel distances to reach such devices due to poor health.

### SUMMARY OF THE INVENTION

Accordingly, as a first aspect of the present invention a method of determining facial metrics of a person is provided. The method comprises: receiving a request that the person requires a mask; navigating an autonomously operable vehicle to the person using at least one imaging device; capturing a number of images of the person using the at least one imaging device; and determining facial metrics of the person from the number of images.

Navigating an autonomously operable vehicle to the person using at least one imaging device may comprise autonomously driving the vehicle to the person.

Capturing a number of images of the person using the at least one imaging device may comprise capturing a sequence of images of the person.

Capturing a number of images of the person using the at least one imaging device may comprise: capturing a first image with the person and the imaging device disposed in a first positioning with respect to each other; and capturing a second image with the person and the imaging device disposed in a second positioning, different from the first positioning, with respect to each other.

Receiving a request that the person requires a mask may comprise receiving an indication via a verbal or physical communication.

Receiving a request that the person requires a custom mask may comprise receiving an indication via an electronic means. Receiving an indication via an electronic means may comprise receiving an indication sent via a smartphone or computer application.

As a second aspect of the present invention, a method of identifying a mask for a person is provided. The method comprises the method of determining facial metrics of a person further comprising: determining a mask for the person from the facial metrics; and identifying the mask to the person.

Identifying the mask to the person may comprise providing the person with a specification of the mask. Identifying the mask to the person may comprise providing the patient with the mask.

The method may further comprise analyzing a test fitment of the mask on the person.

The method may further comprise: determining an unsatisfactory fitment of the mask from the analysis of the test fitment; and performing a corrective action in regard to the mask.

Determining an unsatisfactory fitment of the mask may comprise determining an unsatisfactory amount of leakage; and performing a corrective action may comprise transporting the person, via the autonomously operable vehicle, to another location for a subsequent mask fitment.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is an autonomous vehicle in accordance with an example embodiment of the present invention; and

FIG. 2 is a flowchart showing methods for determining facial metrics of a patient and identifying a mask for the patient in accordance with example embodiments of the present invention.

### DETAILED DESCRIPTION OF EXEMPLARY EMBODIMENTS

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As used herein, the statement that two or more parts or components “engage” one another shall mean that the parts exert a force against one another either directly or through one or more intermediate parts or components. As used herein, the term “number” shall mean one or an integer greater than one (i.e., a plurality).

As used herein, the term “image” shall refer to a representation of the form of a person or thing. Such representation may be a reproduction of the form or may be in the form of electronic information describing the form. As used herein, the term “vehicle” shall refer to a mobile machine that transports people or cargo. Typical vehicles include, for example, without limitation, motorized or electrically powered vehicles such as motorcycles, cars, trucks, and buses. As used herein, the term “autonomous vehicle” or variations thereof shall refer to a vehicle that is capable of sensing its environment and navigating without human input.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

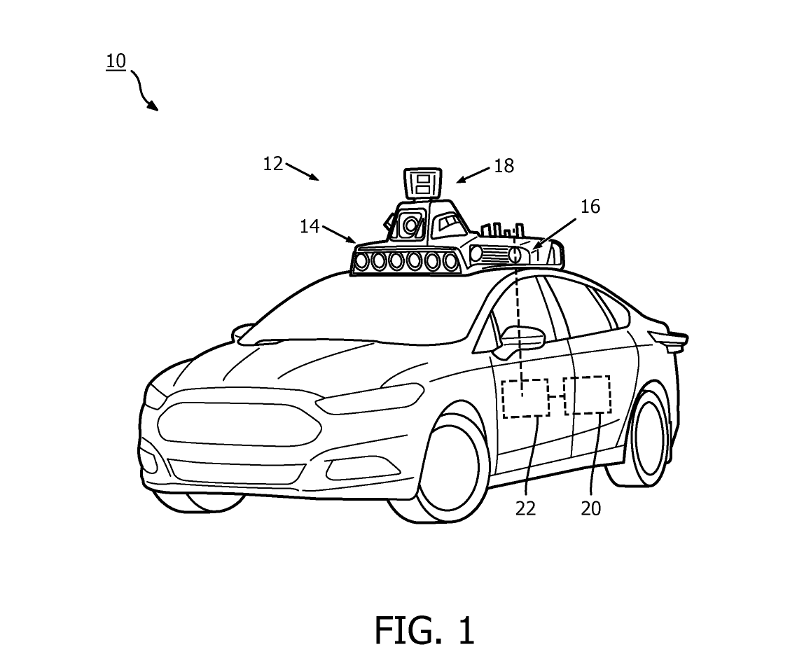

Autonomous vehicles, such as automobiles, one example of which is shown in FIG. 1, are under development and slowly being rolled out for testing in many major cities with great success. As discussed below, autonomous cars have powerful imaging devices and sensors for sensing/imaging the surrounding environment in navigating/operating the vehicle from one location to another. Ultimately these technologies will reach the consumer, meaning that many households will have more powerful than ever scanning and computing technology at their doorsteps and eventually in their garages. Embodiments of the present invention utilize such imaging devices and sensors to provide readily accessible, low-cost solutions for obtaining facial metrics of a patient which may then be used to identify a custom or semi-custom mask for the patient.

Referring to FIG. 1, an example autonomous vehicle 10 in accordance with one example embodiment of the present invention is shown. Autonomous vehicle 10 includes a plurality of imaging devices 12 which provide for autonomous operation of vehicle 10. Imaging devices 12 may include, for example, without limitation: a number of front facing 2-D or 3-D cameras 14, a number of side facing 2-D or 3-D cameras 16, and a roof mounted LiDAR system 18. Data captured by imaging devices 12 is utilized by one or more suitable processing devices 20 in autonomously operating autonomous vehicle 12. As commonly known, during operation of autonomous vehicle 10, the various components of imaging devices 12 are utilized in recognizing and analyzing elements of the surrounding environment in a manner such that autonomous vehicle 10 may navigate from one point to another without incident in a manner similar to that of a manned vehicle. A communication device or devices 22 are further provided in or on autonomous vehicle 10 in communication with processing devices 20 for receiving and transmitting data to or from autonomous vehicle 10. Such communications devices may provide for cellular, Wifi, Bluetooth, or any other suitable communications between autonomous vehicle 10 and the outside world.

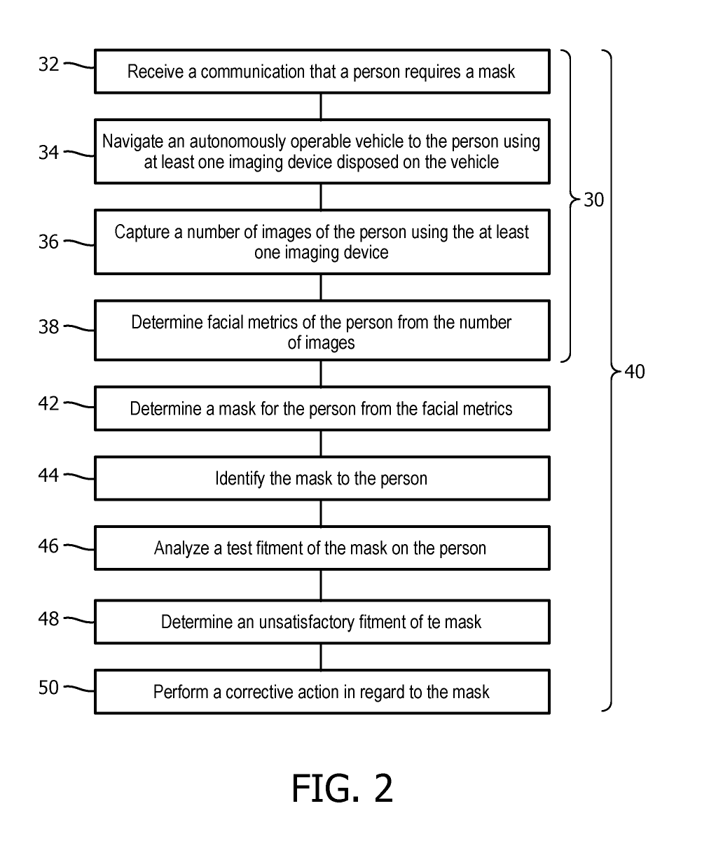

FIG. 2 is a flow chart showing basic steps of a method 30 in accordance with an example embodiment of the present invention, for identifying 3-D facial metrics of a patient. Such method may generally be carried out, for example, without limitation, using an autonomous vehicle 10 such as shown in FIG. 1. Method 30 begins at 32 wherein a communication is received that a person (i.e., a patient) requires a custom (or semi-custom) mask. Such communication may be carried out in a number of ways. For example, such request may be received from a person who is located a distance from autonomous vehicle 10. In such instances, such communication may be carried out via a web-based communication submitted via a website or a smartphone application, or via a phone call (either landline or cellular). As yet another example, such communication may be received from a person who is located in close proximity to autonomous vehicle 10. In such instances, such communication may be carried out in a similar manner as previously discussed or via a local electronic communication, e.g., without limitation, via Bluetooth, Wifi, or other suitable means. Such communication may also be made via non-electronic means such as via a person hailing (similar to a taxi), flagging down or otherwise suitably physically or verbally providing an indication to autonomous vehicle 10.

Once such a communication is received, autonomous vehicle 10 is autonomously navigated to the person (unless already at the person) using imaging devices 12. Once autonomous vehicle 10 has arrived at the person, an interaction with the person and autonomous vehicle 10 may occur in order to confirm the identity of the person. Such confirmation may be carried out via a suitable electronic or non-electronic communication.

Next, as shown at 36, a number of images of the person are captured using at least one imaging device of the plurality of imaging devices 12 that was utilized in navigating autonomous vehicle 10 to the person. In other words, at least one of the devices whose primary function is assisting in autonomously navigating vehicle 10 is utilized as a secondary function to capture images of the person. For example, one or more 3-D scans of the person's face may be recorded using a 3-D imaging device of imaging devices 12. As another example, a plurality of 2-D images may be captured using a single imaging device (with one or both of vehicle 10 and the person moving to slightly different positions) or using two imaging devices functioning in a stereoscopic manner.

After the number of images of the person's face are captured at 36, facial metrics of the person are determined, such as shown at 38, by analyzing the number of images. During such analysis, the number of images may be stitched together and triangulated to construct a 3-D geometry from which a custom CPAP mask for the user may be made or otherwise identified to the user. Alternatively, 2D images could be used to create a 3-D spatial model using any number of other techniques known to one skilled in the art, e.g., for example, without limitation, through the use of disparity maps in the epipolar plane, volumetric deep neural networks (DNN), or generative adversarial network correlations. Alternatively, any suitable analysis method may be employed without varying from the scope of the present invention. The facial metrics of the person determined at 38 may be communicated to the person or to another person or persons for use further use. For example, the facial metrics may be communicated via one or more of a local area network (LAN), a wide area network (WAN), or other suitable means. Alternatively, the determined facial metrics of the person may be employed in a larger method 40 of identifying a custom or semi-custom mask for a user, such as also shown in FIG. 2.

Method 40 includes method 30 as well as further using the determined facial metrics of the person in determining a mask for the person, such as shown at 42. Then, as shown as 44, the mask is then identified and/or provided to the person. As an example, the mask may be identified to the person via information provided to the user, via any suitable form (e.g., electronically or via hardcopy), particularly specifying the mask (i.e., specifications which particularly identify the mask either from amongst other masks or how to construct from scratch or from components). For example, without limitation, a prescription for obtaining a particular mask or a CAD file or similar item containing instructions and/or dimensional information for constructing a custom mask may be provided. Alternatively, the mask may be identified to the person by providing the person with the actual mask, be it custom-made or an off-the-shelf item. In the case of a custom-made mask, a 3-D printer or other suitable automated manufacturing device may be used to provide the mask to the person either generally immediately subsequent to capturing images of the person (i.e., while autonomous vehicle is still with the person) or at a later time (i.e., via special delivery, pick-up from a special location, etc.).

If at 44 a mask is provided to the person, method 40 may further comprise having the person perform a test fitment of the mask with the results being analyzed, such as shown at 46. During such test fitment a number of aspects, e.g., for example, without limitation, seal, comfort, and stability, may be considered. Quantitative results from leak or other testing and/or qualitative results from patient feedback may be considered. If from such fitment and analysis carried out at 46 it is determined that the identified mask is a good fitment for the person, the person may be allowed to take the mask for use in receiving CPAP treatments. However, if from such fitment and analysis carried out at 46 it is determined that the identified mask provides an unsatisfactory fitment with the person, a corrective action in regard to the mask may be performed, such as shown at 50. Such corrective action may be carried out in any of a number of ways without varying from the scope of the present invention. For example, if the fitment is determined to be very poor, steps 36 and 38 may be repeated to determine if incorrect facial metrics of the person were previously determined, thus resulting in identification of an incorrect mask. As another example, if the fitment is determined to be near satisfactory, a new mask, based on previously determined facial metrics may be created or slight adjustments (if possible) made to the previously provided mask may be carried out. As yet another example, if an initial attempt or attempts at fitting the person do not provide suitable results, the person may be transported, via the autonomously operable vehicle, to another location for a subsequent mask fitment.

From the foregoing, it is thus to be appreciated that embodiments of the present invention provide generally low-cost, readily available solutions for providing a high quality custom fit mask to patients.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method of determining facial metrics of a person, the method comprising:
receiving a request that the person requires a mask;
navigating an autonomously operable vehicle to the person using at least one imaging device;
capturing a number of images of the person using the at least one imaging device; and
determining facial metrics of the person from the number of images.

2. The method of claim 1, wherein navigating an autonomously operable vehicle to the person using at least one imaging device comprises autonomously driving the vehicle to the person.

3. The method of claim 1, wherein capturing a number of images of the person using the at least one imaging device comprises capturing a sequence of images of the person.

4. The method of claim 1, wherein capturing a number of images of the person using the at least one imaging device comprises:
capturing a first image with the person and the imaging device disposed in a first positioning with respect to each other; and
capturing a second image with the person and the imaging device disposed in a second positioning, different from the first positioning, with respect to each other.

5. The method of claim 1, wherein receiving a request that the person requires a mask comprises receiving an indication via a verbal or physical communication.

6. The method of claim 1, wherein receiving a request that the person requires a mask comprises receiving an indication via an electronic means.

7. The method of claim 6, wherein receiving an indication via an electronic means comprises receiving an indication sent via a smartphone or computer application.

8. A method of identifying a mask for a person, the method comprising:
(a) determining facial metrics of a person, the method comprising:
receiving a request that the person requires a mask,
navigating an autonomously operable vehicle to the person using at least one imaging device,
capturing a number of images of the person using the at least one imaging device, and
determining facial metrics of the person from the number of images;
(b) determining a mask for the person from the facial metrics; and
(c) identifying the mask to the person.

9. The method of claim 9, wherein identifying the mask to the person comprises providing the person with a specification of the mask.

10. The method of claim 9, wherein identifying the mask to the person comprises providing the patient with the mask.

11. The method of claim 10, further comprising analyzing a test fitment of the mask on the person.

12. The method of claim 11, further comprising:
determining an unsatisfactory fitment of the mask from the analysis of the test fitment; and
performing a corrective action in regard to the mask.

13. The method of claim 12, wherein determining an unsatisfactory fitment of the mask comprises determining an unsatisfactory amount of leakage; and wherein performing a corrective action comprises transporting the person, via the autonomously operable vehicle, to another location for a subsequent mask fitment.
